(19) United States
(12) Patent Application Publication
(10) Pub. No.: US 2008/0161840 A1
(43) Pub. Date: Jul. 3, 2008

(54) ATHERECTOMY METHODS AND APPARATUS

(75) Inventors: Ricardo Osiroff, Ganei-Tikva (IL); Izhak Kirshenbaum, Rosh-Ha'ain (IL); Yaniv Garty, San Francisco, CA (US); Yosi Weitzman, Tel Aviv (IL); Ariel Ben-Porath, Gealia (IL); Yossi Gross, Moshav Mazor (IL)

Correspondence Address: DARBY & DARBY P.C., P.O. BOX 770, Church Street Station, New York, NY, 10008-0770, US

(73) Assignee: VASCURE LTD., Gealia (IL)

(21) Appl. No.: 11/944,266

(22) Filed: Nov. 21, 2007

Related U.S. Application Data
(60) Provisional application No. 60/882,605, filed on Dec. 29, 2006.

(51) Int. Cl. A61B 17/22

(52) U.S. Cl. 606/159

(57) ABSTRACT

Apparatus is provided for removing plaque from a blood vessel of a subject, including a catheter shaped to define an opening that is placed in the blood vessel. A pressure source propels a fluid jet through the opening, and a pressure sensor detects a pressure in the blood vessel induced by the jet. A control unit steers the jet in response to the detected pressure. Other embodiments are also described.

### CROSS-REFERENCES TO RELATED APPLICATIONS

The present application claims priority from U.S. Provisional Patent Application 60/882,605 to Kirshenbaum, filed Dec. 29, 2006, entitled, “Atherectomy Method and Apparatus,” which is incorporated herein by reference.

This application is filed on even date with a PCT patent application, entitled, “Atherectomy methods and apparatus,” which is incorporated herein by reference.

### FIELD OF THE INVENTION

The present invention generally relates to medical apparatus. Specifically, the present invention relates to methods and apparatus for removing vascular plaque.

### BACKGROUND OF THE INVENTION

Vascular plaque is an accumulation of lipids, calcium and other deposits in the wall of a blood vessel. A buildup of plaque in a blood vessel wall can cause the blood vessel to become occluded and may lead to a heart attack, a stroke or peripheral artery disease.

U.S. Pat. No. 5,135,484 to Wright, which is incorporated herein by reference, describes a method and apparatus for removing plaque from vessels by at least partially isolating a portion of a vessel which is partially occluded by plaque from the remainder of the vessel, forcing a slurry to flow in contact with the plaque in the vessel to abrade the plaque, and withdrawing the slurry from the vessel. In an embodiment, the apparatus is described as comprising means for measuring fluid pressure in the vessel and controlling the pumping rate and/or withdrawal rate as a function of the measured pressure.

U.S. Pat. No. 4,729,763 to Henrie, which is incorporated herein by reference, describes a catheter for removing occlusive material from the stenosis of a blood vessel. The catheter comprises a flexible outer tube and a flexible rotatable inner tube which is concentric with and spaced radially inward of the outer tube forming a channel between the two tubes through which liquid is supplied to the vessel. Hollow cutting means are connected to the rotatable inner tube and are positioned within the free end of the catheter to avoid contact of the cutting means with the blood vessel. The fluid flowing in the channel between the tubes to the blood vessel is described as being capable of dissolving and dislodging the stenosis material where it is entrained in the fluid. The entrained stenosis material is cut into smaller pieces and withdrawn as the fluid is sucked through the hollow cutting means and out through the inner tube. The catheter is additionally provided with means for viewing the condition of the blood vessel interior throughout the procedure.

U.S. Pat. No. 4,902,276 to Zakko, which is incorporated herein by reference, describes a fully automatic organ pressure sensitive apparatus for dislodging and removing obstructions in bodily cavities or organs by both delivering and removing fluid thereto, operable by high rate continuous or intermittent infusion of fluid solvent over a set pressure range to effect rapid dissolution and removal of the obstruction without complications to the patient. By continuous feedback monitoring of fluid pressure in the bodily organ or cavity of interest, the apparatus is described as being capable of constantly varying infusion and aspiration rates to maintain the set passages range. If the pressure persists above or below the set range, the apparatus activates a safety feature leading to a period of maximal aspiration and cessation of infusion, followed by cessation of solvent transfer and triggering of an alarm to alert the operator.

U.S. Pat. No. 7,122,017 to Moutafis et al., which is incorporated herein by reference, describes a variety of surgical instruments for forming a liquid jet, which are useful for performing a wide variety of surgical procedures. In some embodiments, surgical liquid jet instruments are provided, the instruments having a pressure lumen and an evacuation lumen, where the pressure lumen includes at least one nozzle for forming a liquid jet and where the evacuation lumen includes a jet-receiving opening for receiving the liquid jet when the instrument is in operation. In some embodiments, the pressure lumen and the evacuation lumen of the surgical liquid jet instruments are constructed and positionable relative to each other so that the liquid comprising the liquid jet, and any tissue or material entrained by the liquid jet can be evacuated through the evacuation lumen without the need for an external source of suction. A variety of surgical liquid jet instruments that are constructed and configured specifically for use in a surrounding liquid environment or a surrounding gaseous environment are also provided. There are also provided a variety of surgical liquid jet instruments that are rotatably deployable from an undeployed position, for insertion into the body of a patient, to a deployed position, in which there is a separation distance between the liquid jet nozzle and the jet-receiving opening that defines a liquid jet path length. Surgical methods utilizing the inventive surgical liquid jet instruments, and methods for forming components of the surgical liquid jet instruments are described.

In an embodiment described in the '017 patent, the liquid pressure supplied to the instrument by the pump or dispenser is variably controllable by an operator of the instrument so that the cutting or ablating power of the liquid jet is adjustable by the operator. This adjustability of the pressure can allow an operator to create a liquid jet with the instrument that is described as being capable of differentiating between different types of tissue within a surgical operating field. For example, a lower pressure can be utilized for cutting or ablating a soft tissue such as fat from a surface of a harder tissue, such as muscle or bone, where the liquid jet has sufficient strength to cut or ablate the soft tissue without damaging the underlying harder tissue. A higher pressure can then be selected that is sufficient to form a liquid jet capable of cutting or ablating hard tissue, such as muscle or bone. In this way, a liquid jet surgical instrument is described as providing highly selective and controllable tissue cutting in various surgical procedures.

U.S. Pat. No. 6,669,710 to Moutafis et al., which is incorporated herein by reference, describes a series of devices useful for surgical procedures utilizing rotatable components for grinding, cutting, ablating, polishing, drilling, screwing, etc., tissues of the body of a patient. The apparatus includes, in one aspect, a series of devices comprising surgical instruments including rotatable shafts, and surgical components drivable by the shafts that can be utilized for contact with tissue in a surgical operating field. Some preferred surgical instruments which are described utilize a liquid jet-driven rotor mechanism for driving rotation of the rotatable shaft. Some preferred instruments include both a liquid jet-driven rotor mechanism and a nozzle at the distal end of the instrument for forming a liquid cutting jet for cutting or ablating tissue of a patient. Such instruments can include a liquid flow directing valve therein that includes a pressure-tight sealing component comprising a sealing element that is constructed and arranged to be slidably movable within a cylinder of the valve. Methods are described for utilizing the surgical instruments in surgical procedures involving both cutting or ablating tissue of a patient with a liquid cutting jet and grinding, cutting, or ablating tissue with a rotating surface of a surgical instrument.

U.S. Pat. No. 5,322,504 to Doherty et al., which is incorporated herein by reference, describes a method and apparatus for the excision and removal of tissue, such as the lens of the eye. The apparatus includes a pencil-like handpiece having a cannula probe extending from the distal end thereof. The probe includes an inner jet tube to direct a high pressure jet of fluid toward a tissue target, and an outer concentric aspiration tube to aspirate and remove fluid and tissue. The jet tube is recessed proximally within the concentric aspiration tube, and the aspiration tube has an end area significantly larger than the end area of the jet tube. These factors are described as cooperating so that the negative pressure exerted by the aspiration tube creates a suction force that offsets and exceeds the force of the fluid jet. The jet tube is connected to a fluid pressure system including a positive displacement pump, a pressure regulator, safety release, control valve, and a pulse former. The jet tube emits pulses of high pressure fluid that impinge reiteratively on the target, which are described as creating shock waves that fracture and emulsify the lens tissue, and the fluid acts as a solvent to transport the emulsified tissue into the aspiration tube. The described handpiece also includes a vacuum bypass port disposed to be selectively occluded by a finger of the surgeon, so that vacuum pressure may be released immediately when needed.

U.S. Pat. No. 5,843,022 to Willard et al., which is incorporated herein by reference, describes an intravascular device and associated system which utilizes pressurized fluid to extract occlusive material. The device is described as being particularly suitable for removing occlusive material which is diffuse, friable, grumous-like, paste-like, granular, and/or chunky. The device includes independently movable fluid input and fluid output tubes and may be advanced over a guide wire. The fluid port holes are located immediately adjacent the distal end of the fluid output tube so as to engage the occlusive material without the need to first traverse the occlusive material with the device. The system utilizes a constant volume pump and associated pressure sensors to maintain balanced flow and immediately detect and correct conditions which may cause clinical complications. In an embodiment, a fluid system for the extraction of vascular occluding material is described, wherein the vasculature has a first pressure zone with a first pressure (P1) proximal to the occluding material, and a second pressure zone with a second pressure (P2) adjacent the occluding material. A control system is described, which controls the pressurized fluid source and the pressurized fluid collector as a function of at least one of the pressures (P1 or P2).

U.S. Pat. No. 5,370,609 to Drasler et al., which is incorporated herein by reference, describes a method of and apparatus for removing a thrombus deposit from the cardiovascular system of a patient without the need to surgically access the location of the thrombus deposit via a cut-down or other surgical procedure. A catheter is inserted percutaneously into the patient at a convenient location either directly or over a previously positioned guide wire. The distal end of the catheter is advanced under fluoroscopy to the site of the thrombus deposit. A balloon is inflated to stabilize the position of the distal end of the catheter within the center of the vessel lumen. A flexible metal tube conveys an extremely high pressure stream of sterile saline solution to at least one jet at the distal end of the catheter. At least one jet positions the thrombus deposit for emulsification by at least one other jet. By directing the jets toward the orifice of the large evacuation lumen of the catheter, a stagnation pressure is induced which propels the emulsion proximally for disposal. The rate of proximal flow of effluent is described as being metered to correspond with the distal flow of saline solution to ensure minimal local impact on the vasculature at the site of the thrombus deposit. Safety monitors turn the system off if one of the lumens or jets becomes clogged. An optional monitor at the distal end of the catheter can monitor power delivery and degree of blockage. An alternative embodiment describes an extra lumen for monitoring of temperature and/or pressure at the site of the thrombectomy.

An article entitled, “Significance of balloon pressure recording during angioplasty. An experimental study,” by Zollicofer et al., Rofo. May 1985;142(5):527-30, which is incorporated herein by reference, describes a study in which pressure and volume of the dilatation balloons were continuously recorded during angioplasty of artificial stenoses, atherosclerotic human cadaver arteries, and normal canine arteries.

The following patents and patent applications, which are incorporated herein by reference, may be of interest:

US Patent Application Publication 2006/0142630 to Meretei

U.S. Pat. No. 4,690,672 to Veltrup

U.S. Pat. No. 6,960,182 to Moutafis

U.S. Pat. No. 6,511,493 to Moutafis

U.S. Pat. No. 5,853,384 to Bair

U.S. Pat. No. 5,735,815 to Bair

U.S. Pat. No. 5,562,692 to Bair

U.S. Pat. No. 6,755,803 to Le et al.

U.S. Pat. No. 6,572,578 to Blanchard

U.S. Pat. No. 5,353,807 to DeMarco

The following articles, which are incorporated herein by reference, may be of interest:

Olbrich et al., “In vivo assessment of coronary artery angioplasty and stent deployment from balloon pressure-volume data,” Physiol Meas. March 2006;27(3):213-23. Epub Jan. 13, 2006

Keris et al., “Biomechanical effects of experimental transluminal angioplasty,” Acta Neurochir (Wien). 1996;138(6):752-8

### SUMMARY OF THE INVENTION

In some embodiments of the present invention, a fluid jet is directed toward plaque in a blood vessel of a subject, in order to abrade the plaque. The fluid pressure that is induced in the blood vessel by the jet is detected, and parameters of the jet are modulated in response to the detected pressure.

Typically, a catheter is inserted into the blood vessel, the distal end of the catheter being shaped to define an opening through which the jet is propelled. The jet is typically directed at plaque within the coronary artery, or within a peripheral artery, of the subject. For some applications, the jet is directed toward occlusive material in a lumen of the subject's body that is not the lumen of a blood vessel, for example, a lumen of the gastrointestinal tract of the subject. In some embodiments, the jet is directed toward plaque on a heart valve of the subject, e.g., the aortic valve.

In some embodiments, a pressure sensor, configured to detect the fluid pressure in the blood vessel induced by the jet, is disposed at the distal end of the catheter. The sensor is configured to move to a plurality of positions within the blood vessel and to detect the pressure induced by the jet at the plurality of positions. In alternative embodiments, a plurality of sensors are disposed at the distal end of the catheter, and the plurality of sensors are configured to detect the pressure at the plurality of positions. Typically, between one and eight, e.g., between one and three, pressure sensors are used.

In response to the detected pressure, a control unit modulates parameters of the jet. For example, the control unit may steer the jet, and/or it may modulate the composition of the jet, the shape of the jet, and/or the temperature of the jet.

The jet is typically directed at the plaque in order to abrade the plaque, i.e., to wear away the plaque by mechanical action. In some embodiments, a low pressure jet is directed toward the plaque and the backpressure of the jet is detected by the pressure sensor, in order to image the blood vessel and the plaque. Subsequently, an abrasive jet, which is of a higher pressure, is directed at the plaque to abrade the plaque. Alternatively or additionally, another therapy for removing the plaque is initiated. Further alternatively or additionally, the plaque is imaged for a different purpose, for example, in order to determine whether or not the plaque requires treatment.

For some applications, the control unit is configured to determine the rate of change of the backpressure of the jet, while the jet is being directed at the plaque. A high rate of change of pressure indicates that the plaque is brittle and that the plaque is being abraded. If the rate of change of pressure is zero or low, it indicates that the jet is impacting the blood vessel wall. An intermediate rate of change of backpressure is an indication that the plaque is being abraded slowly. In some embodiments, the control unit modulates parameters of the jet in response to the detected rate of change of backpressure.

In some embodiments, as an alternative to, or in addition to, determining the pressure in the blood vessel, the control unit is configured to determine the flow velocity distribution in the blood vessel, induced by the jet. For some applications, the one or more pressure sensors disposed at the distal end of the catheter are configured to measure the flow velocity at a plurality of positions within the blood vessel. Alternatively or additionally, one or more Doppler sensors, configured to measure flow velocity within the blood vessel, are disposed at the distal end of the catheter. Typically, the flow velocity sensors are configured to measure the flow velocity in the proximal direction, i.e., the flow velocity of the fluid and plaque debris which have been deflected proximally, toward the catheter. The control unit is configured to image the blood vessel, and/or to modulate parameters of the jet in response to the flow velocity distribution within the blood vessel.

In some embodiments, the control unit is configured to steer the jet in response to the detected pressure and/or flow velocity distribution, induced by the jet in the blood vessel. The control unit is typically configured to steer the jet to direct the jet toward the plaque but not directly toward the blood vessel wall. In some embodiments, the control unit is configured to modulate the rate of the plaque abrasion by steering the jet.

For some applications, the opening through which the jet is propelled comprises a nozzle which is not positioned on the longitudinal axis of the catheter. The control unit is configured to steer the jet by rotating the distal end of the catheter around the longitudinal axis of the catheter. Further alternatively or additionally, the control unit is configured to steer the jet by tilting the distal end of the catheter, or by tilting the nozzle. In embodiments in which the catheter comprises a single pressure sensor, the sensor can be moved to a plurality of positions in the blood vessel, in a similar manner to the above.

For some applications, the opening through which the jet is propelled is steered automatically, using a mechanical self-guiding system. Typically, a hydrodynamic fin is coupled to the distal end of the catheter, the fin being configured to be directed to a point of least pressure, by the backpressure of the jet. The fin is configured to direct the jet toward the plaque by moving to the point of least pressure.

In some embodiments, the control unit is configured to modulate a composition of the jet in response to the detected pressure, the detected rate of change of pressure, and/or other factors. Typically, the jet comprises abrasive particles and the shape, size, concentration, and/or composition of the abrasive particles are modulated.

For some applications, the jet comprises a plurality of types of abrasive particles, each type being configured to dissolve in blood at a respective dissolution rate. The control unit is configured to modulate a dissolution rate of the jet by modulating the composition of the abrasive particles.

In some embodiments, a jet comprising a compound abrasive particle is directed toward plaque in a blood vessel. The compound abrasive particle comprises at least a first and a second layer comprising different respective materials. Typically, the first, inner layer has a high hardness to facilitate abrasion of the blood vessel. The second, outer layer is typically a thin layer configured to slow the dissolution of the inner layer. For example, the inner layer may comprise sodium chloride, and the outer layer, polyglycolic acid. Alternatively, the inner layer may comprise sucrose crystals, and the outer layer, polyvinyl acetate (PVAc). In some embodiments, the compound particle is configured to dissolve at a first rate when the particle is not disposed in contact with blood, and at a second, higher, rate when the particle is disposed in contact with blood.

For some applications, the control unit is configured to modulate non-speed characteristics of the jet, such as the temperature and/or the shape of the jet.

In some embodiments, the temperature of the jet is modulated in order to change a characteristic of the plaque by directing the jet with the modulated temperature toward the plaque.

For some applications, the shape of the nozzle through which the jet is propelled is modulated in order to modulate the shape of the jet. In some embodiments, the shape of the proximal-most portion of the jet is modulated. Alternatively or additionally, the expansion angle of the jet is modulated.

In some embodiments, a balloon is disposed at the distal end of the catheter. For some applications, the balloon is configured to stabilize the catheter and/or to occlude the blood vessel, when the balloon is inflated, to withhold blood flow to the site of the plaque. Alternatively or additionally, a plurality of balloons are disposed in a plurality of positions around the circumference of the distal end of the catheter. The control unit is configured to steer the jet by controlling the inflation of the balloons.

Further alternatively or additionally, a stretch sensor, configured to measure stretching of the blood vessel, is additionally disposed at the distal end of the catheter. A balloon is inflated and the stretch detector measures the resultant stretching of the blood vessel, to determine characteristics of the blood vessel, and/or characteristics of the plaque. In some embodiments, the jet is modulated in response to the detected stretching of the blood vessel.

There is therefore provided in accordance with an embodiment of the present invention, apparatus for removing plaque from a blood vessel of a subject, including:

a catheter shaped to define an opening and configured to be placed in the blood vessel;

a pressure source configured to propel a fluid jet through the opening;

a pressure sensor configured to detect a pressure in the blood vessel induced by the jet; and

a control unit configured to steer the jet in response to the detected pressure.

In an embodiment, the plaque includes plaque in a coronary artery, and the pressure source is configured to propel the jet through the opening toward the plaque in the coronary artery.

In an embodiment, the plaque includes plaque in a peripheral artery, and the pressure source is configured to propel the jet through the opening toward the plaque in the peripheral artery.

In an embodiment, the control unit is configured to identify that the jet is impacting the blood vessel and to steer the jet in response to the identifying.

In an embodiment, the catheter includes a distal end and a metal tip at the distal end, and the apparatus includes a magnetic source, the metal tip being configured to be guided by the magnetic source.

In an embodiment, the control unit is configured to modulate a rate at which the plaque is removed from the blood vessel, by steering the jet.

In an embodiment, the control unit is configured to reduce damage to a wall of the blood vessel by steering the jet.

In an embodiment, the pressure source is configured to abrade the plaque by propelling the jet toward the plaque.

In an embodiment, the pressure source is configured to propel the fluid jet in a direction that is not parallel or perpendicular to a local longitudinal axis of the blood vessel.

In an embodiment, the pressure source is configured to propel a smoothing jet through the opening subsequent to propelling the fluid jet, the fluid jet having a first set of parameters and the smoothing jet having a second set of parameters.

In an embodiment, the pressure source is configured to inhibit restenosis of the blood vessel by propelling the smoothing jet.

In an embodiment, the apparatus includes a cutting tool configured to cut the plaque.

In an embodiment, the cutting tool is configured to be steered in response to the detected pressure.

In an embodiment, the cutting tool is configured to be powered by the jet.

In an embodiment, the cutting tool is configured to terminate cutting the plaque at a cutting termination time that is subsequent to an initiation of the propelling of the jet by the pressure source.

In an embodiment, the cutting tool is configured to terminate cutting the plaque at a cutting termination time that is prior to an initiation of the propelling of the jet by the pressure source.

In an embodiment, the pressure source is configured to terminate propelling the jet at a fluid jet termination time that is subsequent to an initiation of the cutting of the plaque by the cutting tool.

In an embodiment, the pressure source is configured to terminate propelling the jet at a fluid jet termination time that is prior to an initiation of the cutting of the plaque by the cutting tool.

In an embodiment, the sensor is configured to detect a plurality of pressures induced by the jet, at respective positions within the blood vessel.

In an embodiment, the sensor includes a plurality of sensors configured to be disposed at each of the positions within the blood vessel and configured to detect the pressure induced by the jet at each of the positions.

In an embodiment, the sensor is configured to be moved to each of the positions within the blood vessel and to detect the pressure at each of the positions.

In an embodiment, the control unit is configured to steer the jet by steering the opening.

In an embodiment, the catheter includes a distal end, and the control unit is configured to steer the opening by tilting the distal end of the catheter.

In an embodiment, the catheter includes a distal end, the opening is not disposed on a longitudinal axis of the catheter, and the control unit is configured to steer the opening by rotating the distal end of the catheter.

In an embodiment, the apparatus includes a liquid configured to form the jet by being propelled through the opening by the pressure source.

In an embodiment, the liquid includes saline solution.

In an embodiment, the liquid includes a contrast agent.

In an embodiment, the liquid includes a drug.

In an embodiment, the liquid includes frozen particles configured to melt when disposed in contact with blood.

In an embodiment, the liquid includes drug eluting particles.

In an embodiment, the liquid includes abrasive particles.

In an embodiment, the abrasive particles include pH-sensitive particles configured to dissolve when disposed in contact with blood.

In an embodiment, the abrasive particles are shaped to define at least first and second layers thereof, the first and second layers including first and second materials, the first and second materials having different respective dissolution rates in blood.

In an embodiment, the abrasive particles are configured to dissolve at a first dissolution rate when disposed within the catheter and at a second dissolution rate when disposed in contact with blood.

In an embodiment, the abrasive particles are configured to be in a first phase when disposed within the catheter and to be in a second phase when disposed in contact with blood.

In an embodiment, the abrasive particles are configured to be solid when disposed within the catheter and to be liquid when disposed in contact with blood.

In an embodiment, the catheter is configured to direct the jet toward a base of the plaque from an angle of between 0 degrees and 20 degrees from a longitudinal axis of the blood vessel in which the plaque is disposed.

In an embodiment, the catheter is configured to direct the jet toward the base of the plaque from an angle of between 5 degrees and 15 degrees from the longitudinal axis of the blood vessel in which the plaque is disposed.

In an embodiment, the catheter is configured to direct the jet toward a portion of the plaque disposed toward a longitudinal axis of the blood vessel from an angle of between 5 degrees and 30 degrees from a surface of the blood vessel in which the plaque is disposed.

In an embodiment, the catheter is configured to direct the jet toward the portion of the plaque from an angle of between 15 degrees and 25 degrees from the surface.

In an embodiment, the apparatus includes an imaging device configured to image the blood vessel.

In an embodiment, the catheter is shaped to define an imaging lumen, and the imaging device is configured to be advanced into the blood vessel via the imaging lumen.

In an embodiment, the imaging device includes an imaging device selected from the group consisting of: an ultrasound device, and an optical coherent tomography device.

In an embodiment, the imaging device includes between one and eight flow velocity sensors configured to be disposed at a distal end of the catheter and to detect a flow velocity distribution induced by the jet.

In an embodiment, the imaging device includes between one and three flow velocity sensors.

In an embodiment, the imaging device includes an MRI device.

In an embodiment, the catheter includes a distal end which includes an MR-sensitive tip, and the MRI device is configured to monitor progress of the catheter through the blood vessel by imaging the tip.

In an embodiment, the imaging device includes an x-ray detector.

In an embodiment, the catheter includes a distal end which includes a radiopaque tip, and the x-ray detector is configured to monitor progress of the catheter through the blood vessel by imaging the radiopaque tip.

In an embodiment,

the pressure source is configured to propel the fluid jet in alternating first and second phases thereof, the first phase having a first set of parameters and the second phase having a second set of parameters,

the pressure sensor is configured to detect the pressure during the first phase, and

the control unit is configured to steer the jet during the second phase, in response to the sensed pressure during the first phase.

In an embodiment, the pressure source is configured to propel the jet in the first phase at a lower pressure than a pressure at which the pressure source is configured to propel the jet in the second phase.

In an embodiment, the catheter is shaped to define a lumen configured to facilitate removal of contents of the blood vessel from the blood vessel, and the apparatus includes an analysis unit configured to analyze the contents.

In an embodiment, the control unit is configured to modulate the jet in response to the analysis of the contents.

In an embodiment, the apparatus includes a balloon configured to be inflated inside the blood vessel in a vicinity of the plaque.

In an embodiment, the balloon by being in an inflated state is configured to withhold blood flow to a site of the plaque.

In an embodiment, the control unit is configured to steer the jet by controlling a level of inflation of the balloon.

In an embodiment, the balloon is configured to stretch the blood vessel in the vicinity of the plaque, by being inflated inside the blood vessel.

In an embodiment, the apparatus includes a sensor configured to detect the stretching of the blood vessel.

In an embodiment, the control unit is configured to modulate the jet in response to the detection of the stretching.

There is also provided, in accordance with an embodiment of the present invention, apparatus for removing plaque from a blood vessel of a subject, including:

a catheter shaped to define an opening and configured to be placed in the blood vessel;

a pressure source configured to propel a fluid jet through the opening;

a pressure sensor configured to detect a pressure in the blood vessel induced by the jet; and

a control unit configured to modulate a composition of the jet in response to the detected pressure.

In an embodiment, the control unit is configured to receive an input indicating a body part which the blood vessel feeds, and to regulate the composition of the jet in response to the input.

In an embodiment, the control unit is configured to generate plaque debris having a desired characteristic by modulating the jet.

In an embodiment, the control unit is configured to generate plaque debris having a small particle size by modulating the jet.

In an embodiment, the control unit is configured to generate plaque debris having a particle size that is predominantly less than 40 microns.

In an embodiment, the control unit is configured to generate plaque debris having a particle size that is predominantly less than 30 microns.

In an embodiment, the control unit is configured to identify a characteristic of the plaque and to modulate the composition of the jet in response to the identified characteristic of the plaque.

In an embodiment, the control unit is configured to identify the characteristic of the plaque by receiving an input from a healthcare professional.

In an embodiment, the control unit is configured to identify calcified plaque and to modulate the composition of the jet in response to the identification of the calcified plaque.

In an embodiment, the control unit is configured to determine a rate of change of the detected pressure and to modulate an abrasiveness of the jet in response to the determined rate of change of the detected pressure.

In an embodiment, the control unit is configured to increase the abrasiveness of the jet in response to detecting a rate of change of the pressure that is below a desired rate of change of pressure.

In an embodiment, the control unit is configured to decrease the abrasiveness of the jet in response to detecting a rate of change of the pressure that exceeds a desired rate of change of pressure.

In an embodiment, the apparatus includes a liquid configured to form the jet by being propelled through the opening by the pressure source.

In an embodiment, the liquid includes abrasive particles.

In an embodiment, the control unit is configured to modulate the composition of the jet by modulating a concentration of the abrasive particles.

In an embodiment, the control unit is configured to modulate a composition of the jet by modulating a size of the abrasive particles.

In an embodiment, the control unit is configured to modulate a composition of the jet by modulating a shape of the abrasive particles.

In an embodiment, the control unit is configured to modulate the composition of the jet by modulating a composition of the abrasive particles.

In an embodiment, the liquid includes a plurality of types of abrasive particles, each type being configured to dissolve in blood at a respective dissolution rate, and the control unit is configured to modulate a dissolution rate of the jet by modulating the composition of the abrasive particles.

There is also provided, in accordance with an embodiment of the present invention, apparatus for removing plaque from a blood vessel of a subject, including:

a catheter shaped to define an opening and configured to be placed in the blood vessel;

a pressure source configured to propel a fluid jet through the opening;

a pressure sensor configured to detect a pressure in the blood vessel induced by the jet; and

a control unit configured to modulate a non-speed characteristic of the jet in response to the detected pressure.

In an embodiment, the control unit is configured to modulate the jet by modulating a temperature of the jet.

In an embodiment, the pressure source is configured to change a characteristic of the plaque by propelling the jet toward the plaque.

In an embodiment, the control unit is configured to modulate a temperature of the jet.

In an embodiment, the control unit is configured to maintain the temperature of the jet between −4 C and 24 C.

In an embodiment, the control unit is configured to maintain the temperature of the jet between −4 C and +4 C.

In an embodiment, the pressure source is configured to change a level of brittleness of the plaque by propelling the jet toward the plaque.

In an embodiment, the control unit is configured to maintain the temperature of the jet between 50 C and 70 C.

In an embodiment, the control unit is configured to maintain the temperature of the jet between 55 C and 65 C.

In an embodiment, the pressure source is configured to denature the plaque by propelling the jet toward the plaque.

In an embodiment, the control unit is configured to modulate the jet by modulating a shape of the jet.

In an embodiment, the control unit is configured to modulate the shape of the jet by modulating a shape of the opening.

In an embodiment, the control unit is configured to modulate a cross-sectional area of a proximal-most portion of the jet.

In an embodiment, the control unit is configured to modulate an expansion angle of the jet.

There is also provided, in accordance with an embodiment of the present invention, a method for treating plaque in a blood vessel of a subject, including:

directing a fluid jet toward the plaque;

detecting a pressure induced by the jet in the blood vessel; and

steering the jet in response to the detecting.

There is also provided, in accordance with an embodiment of the present invention, method for treating plaque in a blood vessel of a subject, including:

directing a fluid jet toward the plaque;

detecting a pressure induced by the jet in the blood vessel; and

modulating a composition of the jet in response to the detecting.

In an embodiment, the method further includes identifying a body part which the blood vessel feeds and regulating the composition of the jet in response to the identifying.

In an embodiment, identifying the body part includes identifying a body part selected from the group consisting of: a brain, and a heart.

In an embodiment, regulating the composition of the jet in response to the identifying includes generating by the jet plaque debris having a desired characteristic.

In an embodiment, the desired characteristic includes a small size of particles of the plaque debris, and regulating the composition of the jet includes generating by the jet small plaque debris.

There is also provided, in accordance with an embodiment of the present invention, a method for treating plaque in a blood vessel of a subject, including:

directing a fluid jet toward the plaque;

detecting a pressure induced by the jet in the blood vessel; and

modulating a non-speed characteristic of the jet in response to the detecting.

There is also provided, in accordance with an embodiment of the present invention, a method for removing plaque from a blood vessel of a subject, including:

driving a jet through an opening of a catheter; and

steering the jet toward the plaque using a hydrodynamic surface coupled to the catheter.

There is also provided, in accordance with an embodiment of the present invention, apparatus for removing plaque from a blood vessel of a subject, including:

a catheter which is shaped to define an opening and configured to be placed in the blood vessel;

a pressure source configured to propel a jet through the opening; and

a hydrodynamic surface configured to steer the jet in response to pressure in the blood vessel induced by the jet.

There is also provided, in accordance with an embodiment of the present invention, a method for removing plaque from a heart valve of a subject, including:

directing a jet toward the plaque;

detecting a pressure in a vicinity of the heart valve induced by the jet; and

steering the jet in response to the detecting.

In an embodiment, the heart valve includes an aortic valve of the subject, and detecting the pressure in the vicinity of the heart valve includes detecting the pressure in a vicinity of the aortic valve.

There is also provided, in accordance with an embodiment of the present invention, apparatus for removing plaque from a heart valve of a heart of a subject, including:

a catheter shaped to define an opening and configured to be placed in the heart;

a pressure source configured to propel a jet through the opening;

a sensor configured to detect a pressure in a vicinity of the heart valve induced by the jet; and

a control unit configured to steer the jet in response to the detected pressure.

There is also provided, in accordance with an embodiment of the present invention, apparatus for removing plaque from a blood vessel of a subject, including:

a liquid configured to be directed toward the plaque; and

a compound abrasive particle including at least first and second layers including different respective materials, the particle configured to be suspended within the liquid and to abrade the plaque when the liquid is directed toward the plaque.

In an embodiment, the particle is configured to dissolve at a first rate when the particle is not disposed in contact with blood, and at a second, higher, rate when the particle is disposed in contact with blood.

In an embodiment, the first layer includes a hard layer configured to abrade the plaque, the second layer is configured to have a lower dissolution rate than a dissolution rate of the first layer, and the second layer is disposed further from a center of the particle than the first layer.

In an embodiment, the first layer includes sodium chloride, the second layer includes polyglycolic acid, and the second layer is disposed further from a center of the particle than the first layer.

In an embodiment, the first layer includes sucrose, the second layer includes polyvinyl acetate, and the second layer is disposed further from a center of the particle than the first layer.

There is also provided, in accordance with an embodiment of the present invention, a method for removing an occlusion from a body lumen of a subject, including:

directing a jet toward the occlusion;

detecting a pressure induced by the jet in the lumen; and

steering the jet in response to the detecting.

In an embodiment, the occlusion includes an occlusion of a gastrointestinal tract of the subject and directing the jet toward the occlusion includes directing the jet toward the occlusion of the gastrointestinal tract.

There is also provided, in accordance with an embodiment of the present invention, apparatus for removing an occlusion from a body lumen of a subject, including:

a catheter shaped to define an opening and configured to be placed in the lumen;

a pressure source configured to propel a jet through the opening;

a pressure sensor configured to detect a pressure in the lumen induced by the jet; and

a control unit configured to steer the jet in response to the detected pressure.

There is also provided, in accordance with an embodiment of the present invention, a method for treating plaque in a blood vessel of a subject, including:

directing a fluid jet toward the plaque;

detecting a pressure induced by the jet in the blood vessel; and

imaging the plaque by processing the detected pressure.

There is also provided, in accordance with an embodiment of the present invention, a method for treating plaque in a blood vessel of a subject, including:

directing a fluid jet toward the plaque;

detecting a pressure induced by the jet in the blood vessel; and

facilitating a plaque removal therapy using the detected pressure.

There is also provided, in accordance with an embodiment of the present invention, apparatus for removing plaque from a blood vessel of a subject, including:

a catheter shaped to define an opening and configured to be placed in the blood vessel;

a pressure source configured to propel a jet through the opening; and

a Doppler sensor configured to be disposed at a distal end of the catheter and to determine a flow distribution in the blood vessel induced by the jet.

The present invention will be more fully understood from the following detailed description of embodiments thereof, taken together with the drawings, in which:

### BRIEF DESCRIPTION OF THE DRAWINGS

FIG. 1 is a schematic illustration of an atherectomy device, in accordance with an embodiment of the present invention;

FIG. 2 is a schematic illustration of the distal portion of the atherectomy device, in accordance with an embodiment of the present invention;

FIG. 3 is a schematic illustration of the atherectomy device removing plaque from a blood vessel wall, in accordance with an embodiment of the present invention;

FIGS. 4A and 4B are schematic illustrations of the movements by which a jet and/or a pressure sensor of the atherectomy device can be directed to a position of interest, in accordance with an embodiment of the present invention;

FIGS. 5A and 5B are schematic illustrations of a jet being directed toward plaque, in accordance with respective embodiments of the present invention;

FIGS. 6A and 6B are schematic illustrations of a jet being directed toward plaque, in accordance with respective embodiments of the present invention;

FIG. 7 is a block diagram showing a control unit of the atherectomy device, in accordance with an embodiment of the present invention;

FIGS. 8A-C are schematic illustrations of a jet having respective shapes, in accordance with an embodiment of the present invention;

FIGS. 9A and 9B are schematic illustrations of respective views of an atherectomy device comprising a cutting tool, in accordance with an embodiment of the present invention;

FIG. 10 is a schematic illustration of an atherectomy device comprising a cutting tool, in accordance with an embodiment of the present invention;

FIG. 11 is a schematic illustration of an atherectomy device comprising a balloon, in accordance with an embodiment of the present invention;

FIG. 12 is a schematic illustration of an atherectomy device comprising a plurality of balloons, in accordance with an embodiment of the present invention;

FIG. 13 is a schematic illustration of an atherectomy device comprising a hydrodynamic fin, in accordance with an embodiment of the present invention;

FIG. 14 is a schematic illustration of an atherectomy device removing plaque from a heart valve, in accordance with an embodiment of the present invention; and

FIG. 15 is a schematic illustration of a compound particle, in accordance with an embodiment of the present invention.

### DETAILED DESCRIPTION OF EMBODIMENTS

Reference is now made to FIG. 1, which is a schematic illustration of an atherectomy device 20, in accordance with an embodiment of the present invention. Typically, the device comprises a catheter 22 which is advanced into the blood vessel, guided by a guidewire 26. Atherectomy device 20 typically directs a fluid jet toward plaque in a blood vessel of a subject, in order to abrade the plaque. The distal end of catheter 22 typically comprises a distal portion 24 which is movable independently of the rest of the catheter, as described hereinbelow.

The fluid pressure that is induced in the blood vessel by the jet is detected, and parameters of the jet are modulated in response to the detected pressure. Typically, a control unit 28 is configured to control parameters of the jet in response to the detected pressure.

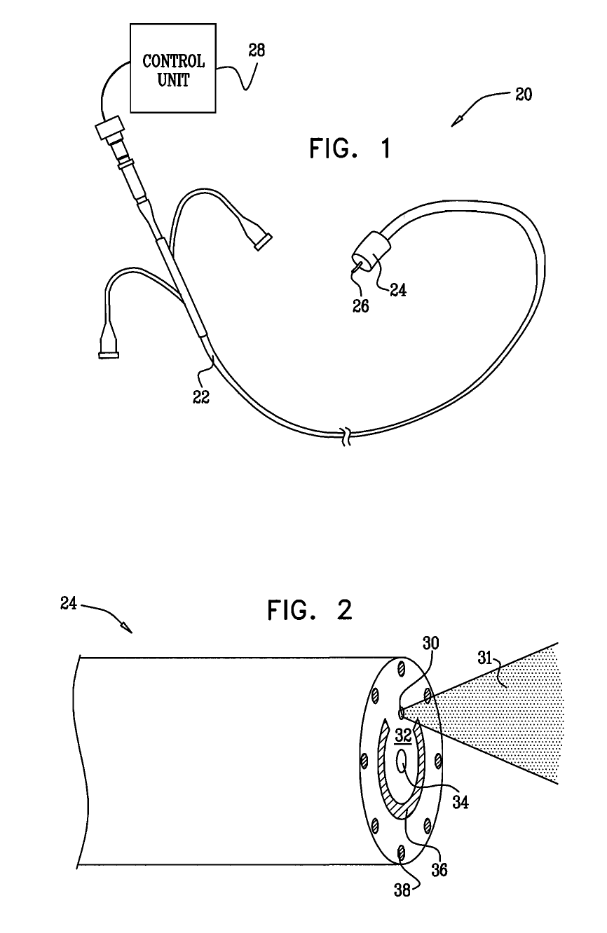

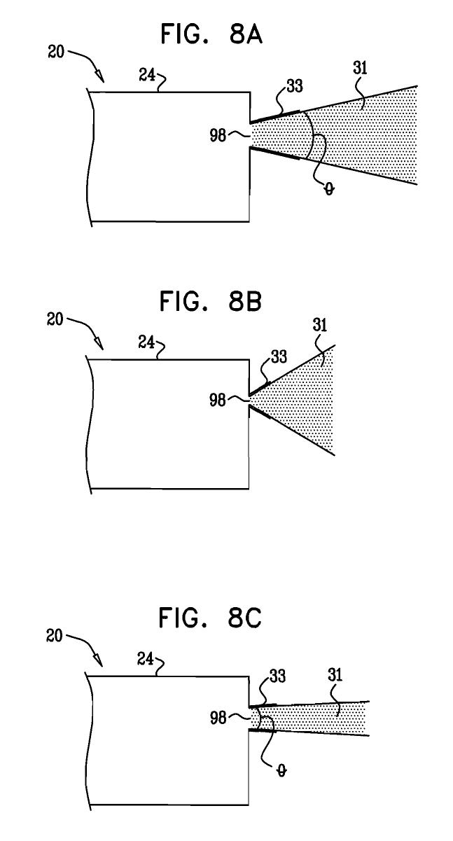

Reference is now made to FIG. 2, which is a schematic illustration of portion 24 of atherectomy device 20, in accordance with an embodiment of the present invention. The distal face 32 of portion 24 is shaped to define a jet opening 30, through which a jet 31 is propelled. Jet opening 30 is typically shaped as a nozzle (as shown in FIGS. 8A-C). The jet is typically directed at plaque in a blood vessel in order to abrade the plaque, i.e., to wear away the plaque by mechanical action. Typically, the jet is directed toward the plaque in a direction that is not parallel or perpendicular with the local longitudinal axis of the blood vessel.

Typically, device 20 comprises a sensor 38, configured to detect pressure induced by jet 31 in the blood vessel into which the jet is being directed. The sensor is configured to be moved to a plurality of positions within the blood vessel and to detect the pressure induced by the jet, at the plurality of positions. In an alternative embodiment (as shown), a plurality of sensors are disposed on the distal face of portion 24, and the plurality of sensors are configured to detect the pressure at the plurality of positions.

In response to the detected pressure, control unit 28 modulates parameters of the jet. For example, the control unit may steer the jet, and/or it may modulate the composition of the jet, the shape of the jet, and/or the temperature of the jet.

Alternatively, or additionally, sensors 38 are configured to measure the flow velocity at a plurality of positions within the blood vessel. Typically, between one and eight, e.g., between one and three, pressure sensors 38 detect pressure, and/or flow velocity, within the blood vessel.

Further alternatively or additionally, one or more Doppler sensors, configured to measure flow velocity within the blood vessel, are disposed on distal portion 24 of catheter 22. Typically, the flow velocity sensors are configured to measure the flow velocity at least in the proximal direction, i.e., the flow velocity of the fluid and plaque debris which have been deflected proximally, toward the catheter.

In some embodiments, control unit 28 is configured to steer the jet in response to the detected pressure and/or flow velocity distribution in the blood vessel, induced by jet 31. The control unit is typically configured to steer the jet to direct the jet toward the plaque but not directly toward the blood vessel wall. In some embodiments, the control unit is configured to modulate the rate of the plaque abrasion by steering the jet. In some embodiments, the control unit steers the jet in order to direct the jet toward asymmetric plaque.

In some embodiments, catheter 22 defines an aspiration channel 36, the opening of which is defined by distal face 32 of portion 24. The aspiration channel is configured to remove the contents of the jet and the plaque debris from the blood vessel. Typically, the backpressure of the jet causes the jet contents and the plaque debris to enter the aspiration channel. For some applications, the plaque debris are analyzed to determine characteristics of the plaque. In some embodiments, control unit 28 analyzes the debris in real-time and modulates parameters of the jet in response to the analysis.

For some applications, the fluid of jet 31 comprises a saline solution that typically includes a suspension of abrasive particles. In some embodiments, the abrasive particles are configured to dissolve at a first rate when the particles are not disposed in contact with blood, and at a second, higher, rate when the particles are disposed in contact with blood. For some applications, the abrasive particles are configured to be in one phase when they are disposed within the catheter and to change to a second phase when they come into contact with blood in the blood vessel. For example, the saline solution may contain frozen saline flakes which are configured to melt and dissolve in the bloodstream when they come into contact with blood. Alternatively or additionally, the particles are pH-sensitive and are configured to dissolve in blood.

In some embodiments, the fluid comprises a contrast agent configured to facilitate imaging of the blood vessel. For some applications, jet 31 contains drug particles for treating the subject, e.g., for treating the blood vessel. Alternatively or additionally, the jet contains drug eluting particles that are configured to elute a drug to the blood vessel.

In some embodiments, jet 31 is directed toward the plaque, and the backpressure of the jet is detected by the pressure sensor, in order to image the blood vessel and the plaque. Subsequently, jet 31 is directed at the plaque, at a greater pressure, to abrade the plaque. Alternatively or additionally, another therapy for removing the plaque is initiated. Further alternatively or additionally, the plaque is imaged for a different purpose, for example, in order to determine whether the plaque requires treatment.

In some embodiments, subsequent to the plaque being abraded by jet 31, the jet is directed toward the blood vessel wall in order to smooth the blood vessel wall. Jet 31 has a different set of parameters during the smoothing phase from during the abrasive phase. For example, in the smoothing phase jet 31 may include abrasive particles which are smaller, have a more rounded shape, and/or are softer than the abrasive particles that are included in the jet during the abrasive phase. Alternatively or additionally, the jet may be applied at a lower velocity, and/or for a longer period of time during the smoothing phase. In the smoothing phase, the jet is typically configured to prevent restenosis of the blood vessel.

For some applications, control unit 28 is configured to determine the rate of change of the backpressure of jet 31, while the jet is being directed at the plaque. A high rate of change of pressure indicates that the plaque is brittle and that the plaque is being abraded. If the rate of change of pressure is zero or low, it indicates that the jet is impacting the blood vessel wall. An intermediate rate of change of backpressure is an indication that the plaque is being abraded slowly.

In some embodiments, the control unit modulates parameters of the jet in response to the detected rate of change of backpressure. For example, if the rate of change of pressure indicates that the jet is impacting the blood vessel wall the control unit may steer the jet away from the wall. Alternatively, if the rate of change of pressure indicates that the plaque is being abraded slowly, the control unit may modulate the composition of the jet to increase the abrasiveness of the jet. In some embodiments, a healthcare professional identifies which type of plaque the jet is abrading, and modulates the composition of the jet accordingly.

In some embodiments, control unit 28 is configured to modulate a composition of jet 31 in response to the detected pressure, the detected rate of change of pressure, and/or other factors. Typically, the jet comprises abrasive particles and the shape, size, concentration, and/or composition of the abrasive particles are modulated.

For some applications, the composition is modulated in response to the control unit identifying that the plaque is of a certain type, such as brittle plaque, as described hereinabove. Alternatively or additionally, the jet composition is modulated in order to regulate the size of the plaque debris. For example, if the blood vessel supplies the heart or the brain, it is often desirable to limit the size of plaque debris. In some embodiments, a healthcare professional modulates the composition of the jet in accordance with the location of the blood vessel into which the jet is directed. Typically, the jet is modulated to generate plaque debris having a particle size that is predominantly less than 40 microns, e.g., predominantly less than 30 microns.

For some applications, jet 31 comprises a plurality of types of abrasive particles, each type being configured to dissolve in blood at a respective dissolution rate. Control unit 28 is configured to modulate a dissolution rate of the jet by modulating the composition of the abrasive particles suspended in the jet.

For some applications, control unit 28 is configured to modulate non-speed characteristics of jet 31, such as the temperature and/or the shape of the jet.

In some embodiments, the temperature of the jet is modulated in order to change a characteristic of the plaque by directing the jet with the modulated temperature toward the plaque. For example, a jet with a temperature that is between −4 C and +24 C (e.g., between −4 C and +4 C) may be directed toward the plaque to facilitate subsequent abrasion by hardening the plaque and making the plaque more brittle. Alternatively or additionally, a jet with a temperature that is between 50 C and 70 C (e.g., between 55 C and 65 C) is directed toward the plaque to denature the plaque.

For some applications, the blood vessel and the plaque are imaged, for example using MR imaging, ultrasound, optical coherent tomography, and/or x-ray imaging of a radiopaque object in the blood vessel.

In some embodiments, catheter 22 defines a lumen 34 disposed along the longitudinal axis of the catheter. Typically guidewire 26 is inserted into the blood vessel via the lumen (as shown in FIG. 1). For some applications, an imaging device is inserted inside the blood vessel transcatheterally, via lumen 34.

In some embodiments, catheter 22 comprises a radiopaque tip, and the advancement of the catheter through the blood vessel is imaged using x-rays. Alternatively or additionally, the catheter tip is MR-sensitive, and its advancement through the blood vessel is monitored using MRI. Further alternatively or additionally, the catheter tip is magnetic, and the catheter is guided through the blood vessel by guiding the tip using a magnet that is external to the subject's body.

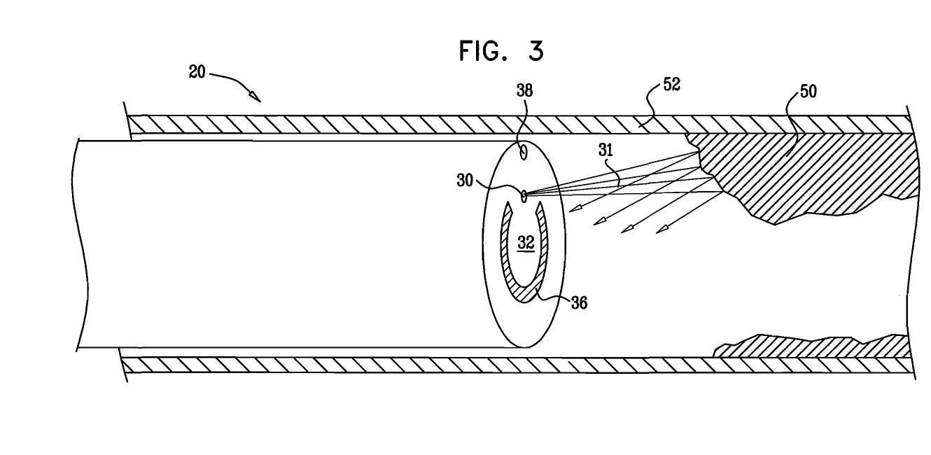

Reference is now made to FIG. 3, which is a schematic illustration of atherectomy device 20 removing plaque 50 from the wall of a blood vessel 52, in accordance with an embodiment of the present invention. Jet 31 is directed at the plaque in order to abrade the plaque. The jet, having impacted the plaque, is deflected toward device 20 in a proximal direction. In the embodiment that is shown, a single pressure sensor 38, which is disposed on the distal face of device 20, detects the backpressure of the jet. In alternative embodiments, as described hereinabove, more than one pressure sensor, and/or flow velocity sensors are used to detect the backpressure and/or the flow velocity.

The jet and plaque debris flowing in the proximal direction enter aspiration channel 36. In some embodiments, the jet and plaque debris are removed from blood vessel 52 using the backpressure of the jet. Alternatively or additionally, a suction source is used to remove the jet and plaque debris from the blood vessel.

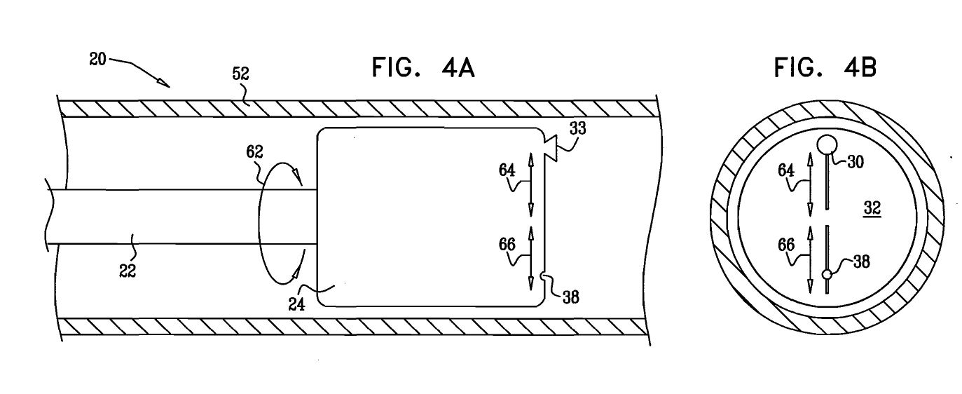

Reference is now made to FIGS. 4A and 4B, which are schematic illustrations of the movements by which jet 31 and/or sensor 38 can be directed, in accordance with an embodiment of the present invention.

In some embodiments (as shown), jet opening 30 is shaped as a nozzle 33 and is disposed at a position on the distal face of catheter 22 that is not on the longitudinal axis of the catheter. The jet can be directed by rotating catheter 22 around its longitudinal axis, for example, in the directions of arrow 62. Alternatively, catheter 22 remains stationary and portion 24 is rotated independently of any rotation of the catheter. In some embodiments, the jet can be directed by tilting portion 24 and/or by tilting nozzle 33.

In some embodiments, opening 30, through which the jet is propelled, can be moved in a radial direction along the distal face 32 of portion 24. For example, opening 30 can be moved in the directions of arrow 64 from a position close to the edge of face 32 to a position that is close to the center of face 32, and vice versa.

In some embodiments, device 30 comprises a sensor 38 which can be moved to a plurality of positions within the blood vessel, as described hereinabove. Typically, the sensor can be moved in a similar manner to the jet opening. Catheter 22, and/or portion 24 can be rotated to move the sensor. Alternatively or additionally, the sensor can be moved by tilting portion 24. Further alternatively or additionally, the sensor can move along face 32 in the directions shown by arrow 66.

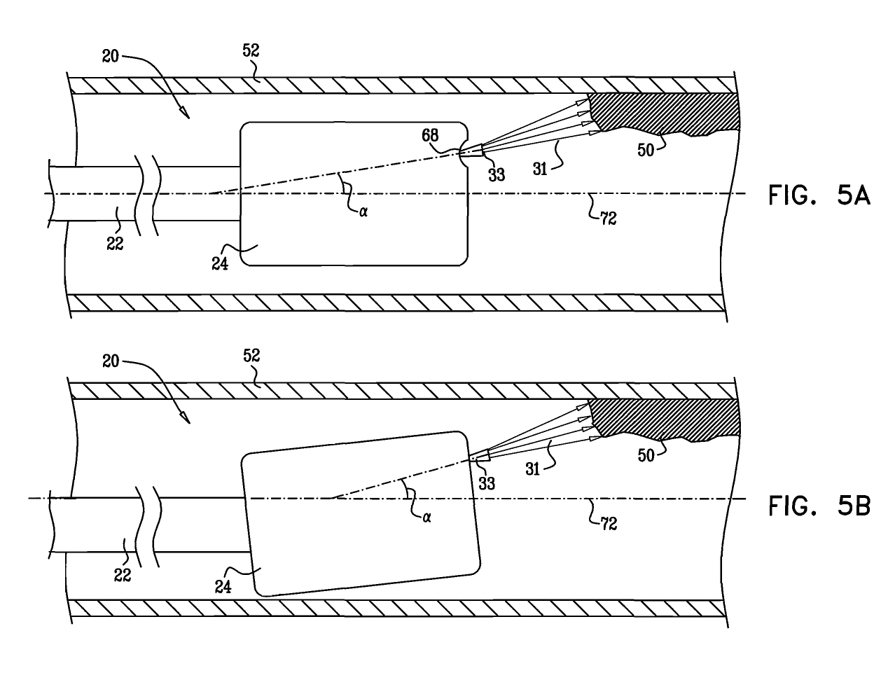

Reference is now made to FIG. 5A, which is a schematic illustration of jet 31 being directed toward plaque 50, by tilting nozzle 33, in accordance with an embodiment of the present invention. As shown, jet 31 is directed towards the base of the plaque (i.e., that portion disposed close to the wall of blood vessel 52). In order to abrade the plaque, the jet is directed toward the plaque at an angle alpha of between 0 degrees and 20 degrees, e.g., between 5 degrees and 15 degrees, from the longitudinal axis 72 of blood vessel 52. Typically, the longitudinal axis of catheter 22 is parallel to longitudinal axis 72 of blood vessel 52, and angle alpha is measured from the longitudinal axis of the catheter.

In some embodiments, a curved track 68 is disposed on distal face 32 of portion 24. Nozzle 33 is tilted by moving the nozzle along the curved track. Alternatively, the nozzle is tilted in a different manner.

Reference is now made to FIG. 5B, which is a schematic illustration of jet 31 being directed toward plaque 50 by tilting distal portion 24, in accordance with an embodiment of the present invention. As shown, jet 31 is directed towards the base of the plaque (i.e., that portion disposed close to the wall of blood vessel 52). In order to abrade the plaque, the jet is directed toward the plaque at an angle alpha, angle alpha being as described with reference to FIG. 5A.

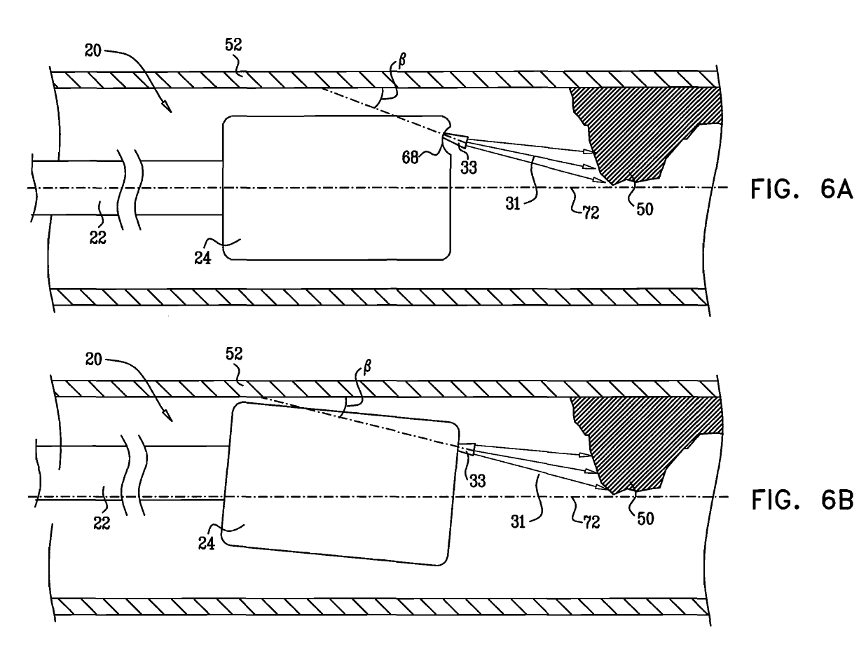

Reference is now made to FIG. 6A, which is a schematic illustration of jet 31 being directed toward plaque 50, by tilting nozzle 33, in accordance with an embodiment of the present invention. As shown in the figure, a portion of the plaque is disposed toward longitudinal axis 72 of blood vessel 52. In order to abrade the plaque, the jet is directed toward the plaque at an angle beta of between 5 degrees and 30 degrees, e.g., between 15 degrees and 25 degrees, from the wall of blood vessel 52.

Reference is now made to FIG. 6B, which is a schematic illustration of jet 31 being directed toward plaque 50 by tilting distal portion 24, in accordance with an embodiment of the present invention. Jet 31 is directed toward the portion of the plaque disposed toward longitudinal axis 72 of blood vessel 52. In order to abrade the plaque, the jet is directed toward the plaque at an angle beta, angle beta being as described with reference to FIG. 6A.

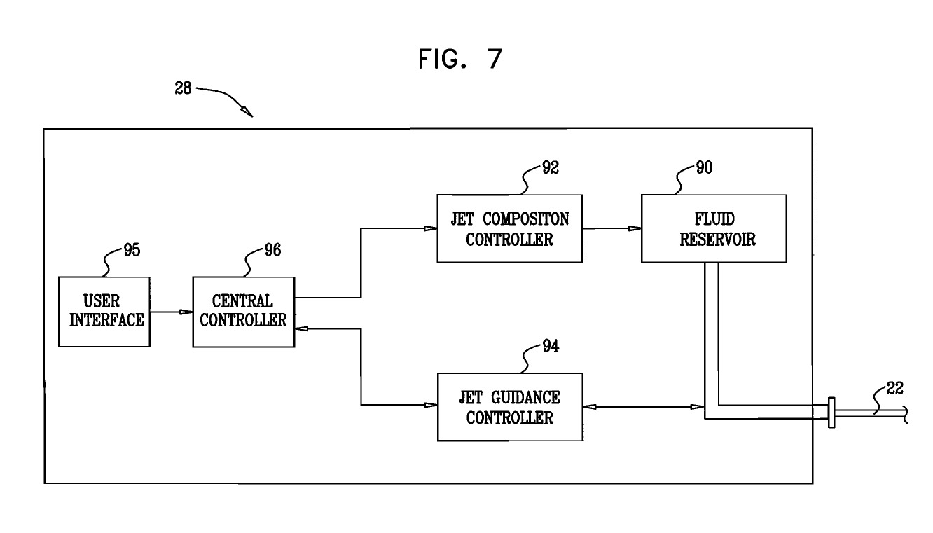

Reference is now made to FIG. 7 which is a block diagram showing control unit 28 of atherectomy device 20, in accordance with an embodiment of the present invention. The control unit is coupled to catheter 22 of the atherectomy device.

In some embodiments, a central controller 96 receives input from sensors 38 disposed at the distal end of catheter 22. In response to the input, the control unit modulates parameters of jet 31, as described hereinabove.

In some embodiments, control unit 28 comprises a user interface 95. For some applications, a healthcare professional can control parameters of the jet via the user interface. Typically, the user interface comprises a screen on which an image or representation of the blood vessel and the plaque are displayed. Alternatively or additionally, the screen may be used to display parameters of the jet.

Typically, in response to an input, central controller 96 directs a signal to a jet composition controller 92, and/or to a jet guidance controller 94. The jet composition controller modulates the composition and/or temperature of fluid in a reservoir 90. (The fluid typically comprises a suspension of abrasive particles in saline solution, as described hereinabove.) The jet guidance controller steers the jet, and/or changes the shape of the jet, as described hereinabove. The modulated fluid is fed from reservoir 90 into catheter 22, and the jet is directed toward plaque in the blood vessel.

Reference is now made to FIGS. 8A-C which are schematic illustrations of jet 31 having respective shapes, in accordance with respective embodiments of the present invention.

For some applications, the shape of jet 31 is modulated, typically in response to pressure detected by the pressure sensor, as described hereinabove. In some embodiments the shape, e.g., the cross-sectional area of the proximal-most portion 98 of the jet is modulated. The cross-sectional area of the proximal-most portion of the jet is greater in FIG. 8A than in FIG. 8B. Alternatively or additionally, the expansion angle theta of the jet is modulated. FIG. 8A shows jet 31 having a greater expansion angle than the expansion angle of jet 31 in FIG. 8C. Typically, the cross-sectional area and/or the angle is controlled by regulating the physical shape of jet nozzle 33.

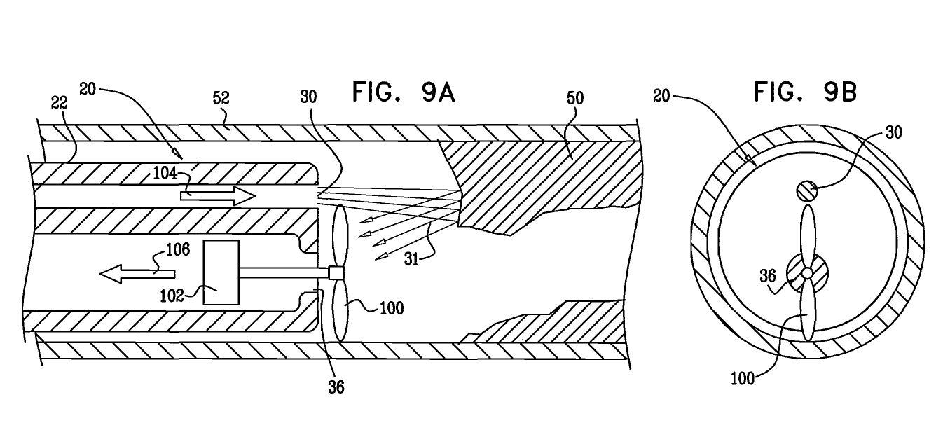

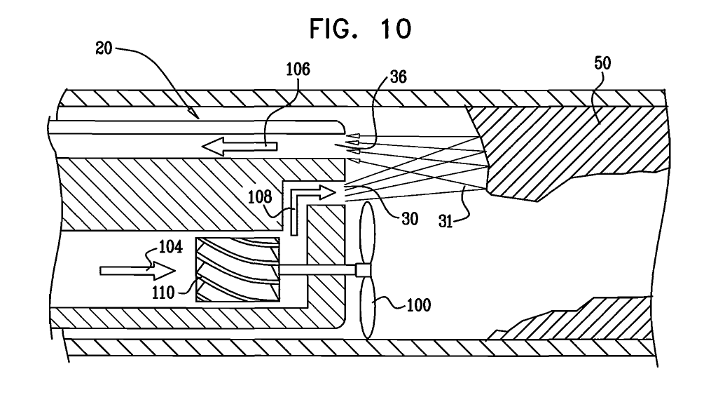

Reference is now made to FIGS. 9A and 9B, which are schematic illustrations of respective views of atherectomy device 20 comprising a cutting tool 100, in accordance with an embodiment of the present invention. Cutting tool 100, which is configured to cut plaque 50, is disposed at the distal end of the catheter. In some embodiments, the cutting tool is powered by a motor 102. For some applications, the cutting tool is steered in response to detected pressure within the blood vessel. Typically, the cutting tool is steered toward the plaque. Further typically, the cutting tool cuts the plaque prior to the jet being directed toward the plaque.

For some applications, cutting tool 100 operates at the same time as jet 31 is directed toward plaque 50 in blood vessel 52. Fluid flows in the direction of arrow 104, through opening 30 toward the plaque. The fluid and plaque debris flow back toward aspiration channel 36. At the same time, cutting tool 100 cuts plaque 50, and the plaque debris from the cutting are washed into channel 36 by the backflow of the jet. The backflow of the jet and the plaque debris flow through channel 36, in the direction of arrow 106, toward the proximal end of catheter 22. Alternatively, cutting tool 100 operates after jet 31 is directed toward plaque 50.

Reference is now made to FIG. 10, which is a schematic illustration of atherectomy device 20 comprising cutting tool 100, in accordance with an embodiment of the present invention. In some embodiments (as shown), the cutting tool is configured to be powered by the fluid pressure of fluid flowing toward opening 30.

Cutting tool 100 is coupled to a rotatable drum 110, the drum comprising vanes. The fluid flows past drum 110 in the direction of arrow 104, causing the drum and cutting tool 100 to rotate. The fluid flows in the direction of arrow 108 and out of opening 30. Typically, jet 31 abrades plaque 50, before, after, or at the same time as the cutting tool cuts the plaque. The jet and the plaque debris flow out of the blood vessel, through aspiration channel 36, in the direction of arrow 106.

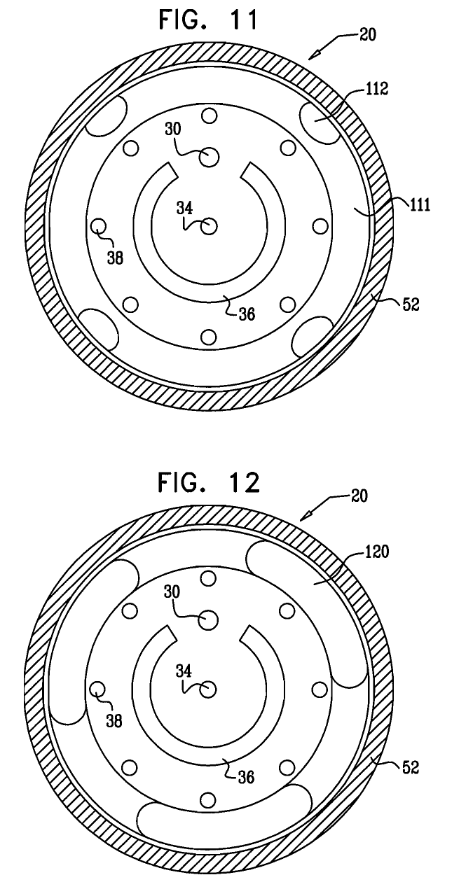

Reference is now made to FIG. 11, which is a schematic illustration of atherectomy device 20 comprising a balloon 111, in accordance with an embodiment of the present invention. Balloon 111 is disposed around the circumference of the distal end of catheter 22. Typically balloon 111 stabilizes the distal end of catheter 22 while jet 31 is being directed toward plaque.

For some applications, balloon 111 is configured to occlude the blood vessel, when the balloon is inflated, to withhold blood flow to the site of the plaque. Alternatively or additionally, one or more stretch sensors 112, configured to measure stretching of the blood vessel, are additionally disposed at the distal end of the catheter. For example, sensors 112 may be configured to detect stretching of the blood vessel by measuring the distances between respective sensors, the sensors being coupled to the balloon, as shown. For some applications, the sensors comprise ultrasound sensors, or other devices known in the art for distance determination. The balloon is inflated and the stretch detectors measure the resultant stretching of the blood vessel, to determine characteristics of the blood vessel, and/or characteristics of the plaque. For some applications, stretching of the blood vessel is measured by recording the pressure and the volume of the balloons during inflation, in accordance with the Zollicofer article cited hereinabove. In some embodiments, the jet is modulated in response to the detected stretching of the blood vessel.

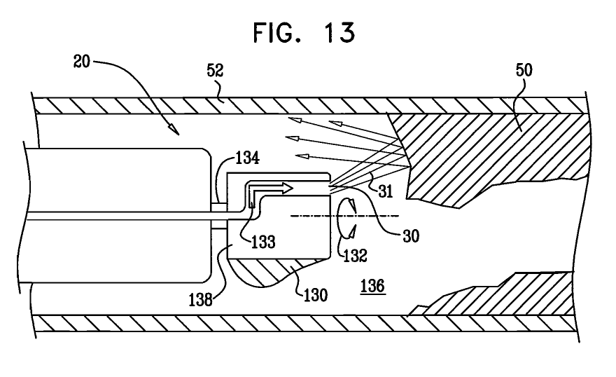

Reference is now made to FIG. 12, which is a schematic illustration of atherectomy device 20 comprising one or more balloons 120, in accordance with an embodiment of the present invention. Typically, the balloons are disposed in a plurality of positions around the circumference of the distal end of catheter 22. The control unit is configured to steer the jet by inflating and deflating the one or more balloons.

Reference is now made to FIG. 13, which is a schematic illustration of atherectomy device 20 comprising a hydrodynamic fin 130, in accordance with an embodiment of the present invention. Fluid flows toward opening 30, in the direction of arrow 133. Jet 31 is directed through opening 30 toward plaque 50. Opening 30 is defined by the distal end of a rotatable drum 138 that rotates around swivel 134. Fin 130 is disposed on the circumference of drum 138. The fin is configured to rotate (e.g., in the directions of arrow 132) away from the high pressure region indicated by the arrows of jet 31, toward a region of low pressure 136, where the backpressure of jet 31 is minimal. The fin is disposed on the drum such that the movement of the fin to region 136 directs jet 31 toward the plaque, as shown in the figure. In all other aspects, device 20 is generally as described hereinabove. Generally, as plaque 50 is eroded by jet 31, the dynamics of the fluid flow around fin 130 change, resulting in region of low pressure 136 moving to a different area, thereby steering fin 130 and jet 31 so that jet 31 is directed toward the new region of greatest protrusion of plaque 50.

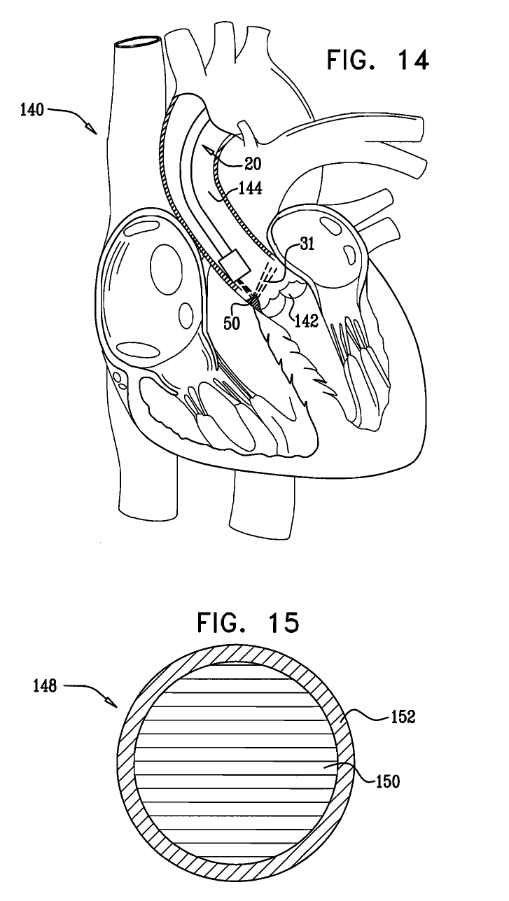

Reference is now made to FIG. 14, which is a schematic illustration of atherectomy device 20 removing plaque 50 from an aortic valve 142 of a subject, in accordance with an embodiment of the present invention. The device is typically advanced toward the subject's heart 140, via the subject's aorta 144, and directs jet 31 toward the plaque to abrade the plaque. In all other aspects, device 20 is generally as described hereinabove. Although the aortic valve is shown in the figures, it is to be understood that the scope of the present invention includes using apparatus described herein to treat other cardiac valves or non-cardiac valves of a subject (e.g., venous valves in the legs of a patient).

Reference is now made to FIG. 15, which is a schematic illustration of a compound abrasive particle 148, in accordance with an embodiment of the present invention. The compound abrasive particle comprises at least a first layer 150 and a second layer 152, comprising different respective materials. Typically inner layer 150 has a high hardness to facilitate abrasion of the blood vessel. Outer layer 152 is typically a thin layer configured to slow the dissolution of the inner layer. For example, the inner layer may comprise sodium chloride, and the outer layer, polyglycolic acid. Alternatively, the inner layer may comprise sucrose crystals, and the outer layer, polyvinyl acetate (PVAc). In some embodiments, the compound particle is configured to dissolve at a first rate when the particle is not disposed in contact with blood, and at a second, higher, rate when the particle is disposed in contact with blood.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for removing plaque from a blood vessel of a subject, comprising:
a catheter shaped to define an opening and configured to be placed in the blood vessel;
a pressure source configured to propel a fluid jet through the opening;
a pressure sensor configured to detect a pressure in the blood vessel induced by the jet; and
a control unit configured to steer the jet in response to the detected pressure.

2-3. (canceled)

4. The apparatus according to claim 1, wherein the control unit is configured to identify that the jet is impacting the blood vessel and to steer the jet in response to the identifying.

5. (canceled)

6. The apparatus according to claim 1, wherein the control unit is configured to modulate a rate at which the plaque is removed from the blood vessel, by steering the jet.

7. The apparatus according to claim 1, wherein the control unit is configured to reduce damage to a wall of the blood vessel by steering the jet.

8. The apparatus according to claim 1, wherein the pressure source is configured to abrade the plaque by propelling the jet toward the plaque.

9. The apparatus according to claim 1, wherein the pressure source is configured to propel the fluid jet in a direction that is not parallel or perpendicular to a local longitudinal axis of the blood vessel.

10-11. (canceled)

12. The apparatus according to claim 1, comprising a cutting tool configured to cut the plaque.

13. The apparatus according to claim 12, wherein the cutting tool is configured to be steered in response to the detected pressure.

14. The apparatus according to claim 12, wherein the cutting tool is configured to be powered by the jet.

15. (canceled)

16. The apparatus according to claim 12, wherein the cutting tool is configured to terminate cutting the plaque at a cutting termination time that is prior to an initiation of the propelling of the jet by the pressure source.

17-18. (canceled)

19. The apparatus according to claim 1, wherein the sensor is configured to detect a plurality of pressures induced by the jet, at respective positions within the blood vessel.

20. The apparatus according to claim 19, wherein the sensor comprises a plurality of sensors configured to be disposed at each of the positions within the blood vessel and configured to detect the pressure induced by the jet at each of the positions.

21. The apparatus according to claim 19, wherein the sensor is configured to be moved to each of the positions within the blood vessel and to detect the pressure at each of the positions.

22. The apparatus according to claim 1, wherein the control unit is configured to steer the jet by steering the opening.

23-24. (canceled)

25. The apparatus according to claim 1, comprising a liquid configured to form the jet by being propelled through the opening by the pressure source.

26-30. (canceled)

31. The apparatus according to claim 25, wherein the liquid comprises abrasive particles.

32-40. (canceled)

41. The apparatus according to claim 1, comprising an imaging device configured to image the blood vessel.

42-43. (canceled)

44. The apparatus according to claim 41, wherein the imaging device comprises between one and eight flow velocity sensors configured to be disposed at a distal end of the catheter and to detect a flow velocity distribution induced by the jet.

45. The apparatus according to claim 44, wherein the imaging device comprises between one and three flow velocity sensors.

46-49. (canceled)

50. The apparatus according to claim 1,
wherein the pressure source is configured to propel the fluid jet in alternating first and second phases thereof, the first phase having a first set of parameters and the second phase having a second set of parameters,
wherein the pressure sensor is configured to detect the pressure during the first phase, and
wherein the control unit is configured to steer the jet during the second phase, in response to the sensed pressure during the first phase.

51-59. (canceled)

60. Apparatus for removing plaque from a blood vessel of a subject, comprising:
a catheter shaped to define an opening and configured to be placed in the blood vessel;
a pressure source configured to propel a fluid jet through the opening;
a pressure sensor configured to detect a pressure in the blood vessel induced by the jet; and
a control unit configured to modulate a composition of the jet in response to the detected pressure.

61. The apparatus according to claim 60, wherein the control unit is configured to receive an input indicating a body part which the blood vessel feeds, and to regulate the composition of the jet in response to the input.

62. The apparatus according to claim 60, wherein the control unit is configured to generate plaque debris having a desired characteristic by modulating the jet.

63-65. (canceled)

66. The apparatus according to claim 60, wherein the control unit is configured to identify a characteristic of the plaque and to modulate the composition of the jet in response to the identified characteristic of the plaque.

67-68. (canceled)

69. The apparatus according to claim 60, wherein the control unit is configured to determine a rate of change of the detected pressure and to modulate an abrasiveness of the jet in response to the determined rate of change of the detected pressure.

70. The apparatus according to claim 69, wherein the control unit is configured to increase the abrasiveness of the jet in response to detecting a rate of change of the pressure that is below a desired rate of change of pressure.

71. The apparatus according to claim 69, wherein the control unit is configured to decrease the abrasiveness of the jet in response to detecting a rate of change of the pressure that exceeds a desired rate of change of pressure.

72. The apparatus according to claim 60, comprising a liquid configured to form the jet by being propelled through the opening by the pressure source.

73. The apparatus according to claim 72, wherein the liquid comprises abrasive particles.

74. The apparatus according to claim 73, wherein the control unit is configured to modulate the composition of the jet by modulating a concentration of the abrasive particles.

75. The apparatus according to claim 73, wherein the control unit is configured to modulate a composition of the jet by modulating a size of the abrasive particles.

76. The apparatus according to claim 73, wherein the control unit is configured to modulate a composition of the jet by modulating a shape of the abrasive particles.

77. The apparatus according to claim 73, wherein the control unit is configured to modulate the composition of the jet by modulating a composition of the abrasive particles.

78. (canceled)

79. Apparatus for removing plaque from a blood vessel of a subject, comprising:
a catheter shaped to define an opening and configured to be placed in the blood vessel;
a pressure source configured to propel a fluid jet through the opening;
a pressure sensor configured to detect a pressure in the blood vessel induced by the jet; and
a control unit configured to modulate a non-speed characteristic of the jet in response to the detected pressure.

80. The apparatus according to claim 79, wherein the control unit is configured to modulate the jet by modulating a temperature of the jet.

81-88. (canceled)

89. The apparatus according to claim 79, wherein the control unit is configured to modulate the jet by modulating a shape of the jet.

90-92. (canceled)

93. A method for treating plaque in a blood vessel of a subject, comprising:
directing a fluid jet toward the plaque;
detecting a pressure induced by the jet in the blood vessel; and
steering the jet in response to the detecting.

94-147. (canceled)

148. A method for treating plaque in a blood vessel of a subject, comprising:
directing a fluid jet toward the plaque;
detecting a pressure induced by the jet in the blood vessel; and
modulating a composition of the jet in response to the detecting.

149-169. (canceled)

170. A method for treating plaque in a blood vessel of a subject, comprising:
directing a fluid jet toward the plaque;
detecting a pressure induced by the jet in the blood vessel; and
modulating a non-speed characteristic of the jet in response to the detecting.

171-182. (canceled)

183. A method for removing plaque from a blood vessel of a subject, comprising:
driving a jet through an opening of a catheter; and
steering the jet toward the plaque using a hydrodynamic surface coupled to the catheter.

184. Apparatus for removing plaque from a blood vessel of a subject, comprising:
a catheter which is shaped to define an opening and configured to be placed in the blood vessel;
a pressure source configured to propel a jet through the opening; and
a hydrodynamic surface configured to steer the jet in response to pressure in the blood vessel induced by the jet.

185. A method for removing plaque from a heart valve of a subject, comprising:
directing a jet toward the plaque;
detecting a pressure in a vicinity of the heart valve induced by the jet; and
steering the jet in response to the detecting.

186. (canceled)

187. Apparatus for removing plaque from a heart valve of a heart of a subject, comprising:
a catheter shaped to define an opening and configured to be placed in the heart;
a pressure source configured to propel a jet through the opening;
a sensor configured to detect a pressure in a vicinity of the heart valve induced by the jet; and
a control unit configured to steer the jet in response to the detected pressure.

188. (canceled)

189. Apparatus for removing plaque from a blood vessel of a subject, comprising:
a liquid configured to be directed toward the plaque; and
a compound abrasive particle comprising at least first and second layers comprising different respective materials, the particle configured to be suspended within the liquid and to abrade the plaque when the liquid is directed toward the plaque.

190-193. (canceled)

194. A method for removing an occlusion from a body lumen of a subject, comprising:
directing a jet toward the occlusion;
detecting a pressure induced by the jet in the lumen; and
steering the jet in response to the detecting.

195. (canceled)

196. Apparatus for removing an occlusion from a body lumen of a subject, comprising:
a catheter shaped to define an opening and configured to be placed in the lumen;
a pressure source configured to propel a jet through the opening;
a pressure sensor configured to detect a pressure in the lumen induced by the jet; and
a control unit configured to steer the jet in response to the detected pressure.

197. A method for treating plaque in a blood vessel of a subject, comprising:
directing a fluid jet toward the plaque;
detecting a pressure induced by the jet in the blood vessel; and
imaging the plaque by processing the detected pressure.

198. A method for treating plaque in a blood vessel of a subject, comprising:
directing a fluid jet toward the plaque;
detecting a pressure induced by the jet in the blood vessel; and
facilitating a plaque removal therapy using the detected pressure.

199. Apparatus for removing plaque from a blood vessel of a subject, comprising:
a catheter shaped to define an opening and configured to be placed in the blood vessel;
a pressure source configured to propel a jet through the opening; and
a Doppler sensor configured to be disposed at a distal end of the catheter and to determine a flow distribution in the blood vessel induced by the jet.
